(19) United States
(12) Patent Application Publication
(10) Pub. No.: US 2020/0324042 A1
(43) Pub. Date: Oct. 15, 2020

(54) WEARABLE FLUID DELIVERY SYSTEM

(71) Applicant: MedUX LLC, Syracuse, NY (US)

(72) Inventors: Quinn M. King, Yarmouth, ME (US); Alec P. Gillinder, Upper Saddle River, NJ (US)

(21) Appl. No.: 16/836,012

(22) Filed: Mar. 31, 2020

Related U.S. Application Data
(60) Provisional application No. 62/833,230, filed on Apr. 12, 2019.

(51) Int. Cl. A61M 5/142

(52) U.S. Cl. CPC A61M 5/1424; A61M 2005/14272; A61M 2209/088; A61M 2210/083

(57) ABSTRACT

A universal wearable fluid delivery system includes a body panel which includes a plurality of connection points. A fluid dispenser includes a taut pocket defined by at least one taut surface and a portion of a surface of the body panel and is adapted to receive a fluid bag. A stretch pocket is disposed within the taut pocket. The stretch pocket is defined between a surface of at least one stretch panel and about a common portion of the surface of the body panel and is adapted to receive a pressure infusion bag disposed within. At least one strap is coupled to the body panel via two connection points of the plurality of connection points to secure the fluid dispenser to a body of a human or animal patient. A wearable fluid delivery system and a fluid dispenser and body panel assembly are also described.

### CROSS-REFERENCE TO RELATED APPLICATIONS

This application claims priority to and the benefit of co-pending U.S. provisional patent application Ser. No. 62/833,230, WEARABLE PORTABLE IV SYSTEM, filed Apr. 12, 2019, which application is incorporated herein by reference in its entirety.

### FIELD OF THE APPLICATION

The application relates to a fluid delivery system, particularly to a wearable fluid delivery system.

### BACKGROUND

Fluid delivery therapy, including intravenous (IV) therapy, is the infusion of a liquid-based medication, or other substance, into the body of an animal or a human patient. Intravenous (IV) therapy is the infusion of a liquid-based medication, or other substance, directly into a vein of a patient. An IV delivery system is often used to facilitate intravenous therapy. From 2005-2014 there was an approximate average of 380 disasters and there are 76,000 deaths from disasters annually. Medical rescue operatives not only use gravity fed IV systems to hydrate and restore electrolytes in their patients, but they also will often use pressure infuser bags to pump high amounts of fluid into a patient to raise their blood pressure, get oxygen to the central organs and brain, and so that the patient can maintain a stabilized condition until reaching the hospital.

### SUMMARY

A universal wearable fluid delivery system includes a body panel. The body panel includes a plurality of connection points. A fluid dispenser includes a taut pocket defined by at least one taut surface and a portion of a surface of the body panel. The taut pocket is adapted to receive a fluid bag. A stretch pocket is disposed within the taut pocket. The stretch pocket is defined between a surface of at least one stretch panel and about a common portion of the surface of the body panel. The stretch pocket is adapted to receive a pressure infusion bag disposed within. At least one strap is coupled to the body panel via two connection points of the plurality of connection points. The at least one strap is to secure the fluid dispenser to a body of a human or animal patient.

The universal wearable fluid delivery system can be used in a variety of different strap configurations such as the exemplary configurations which follow.

The universal wearable fluid delivery system can include at a first end of the body panel, a first connection point to the body panel and a second connection point to the body panel, the first connection point and the second connection point are mechanically coupled to an adjustable removable across a chest and under arm strap, and at a second end of the body panel, and opposite to the first end, an above the elbow strap.

The universal wearable fluid delivery system can include about midway between a first end and a second end of the body panel, a third connection point to the body panel and a fourth connection point to the body panel, the third connection point and the fourth connection point are mechanically coupled to an adjustable bicep strap, and at a second end of the body panel, opposite to the first end, an above the elbow strap.

The universal wearable fluid delivery system can include at a first end of the body panel, a first connection point to the body panel and a second connection point to the body panel, the first connection point and the second connection point are mechanically coupled to an adjustable removable waist strap, and at the second end of the body panel, opposite to the first end, an above the knee strap.

The universal wearable fluid delivery system can include about midway between a first end and a second end of the body panel, a third connection point to the body panel and a fourth connection point to the body panel, the third connection point and the fourth connection point are mechanically coupled to an adjustable removable upper leg or crotch strap, and at the second end of the body panel, opposite to the first end, an above the knee strap.

The universal wearable fluid delivery system can include a first end of the body panel, at a first end of the body panel, a first connection point to the body panel and a second connection point to the body panel, the first connection point and the second connection point are mechanically coupled to an adjustable removable around a neck strap, and at a second end of the body panel, and about opposite to the first end, a lower torso or waist strap.

The universal wearable fluid delivery system can include a first end of the body panel, a first connection point to the body panel and a second connection point to the body panel, the first connection point and the second connection point are mechanically coupled to an adjustable removable over a shoulder forechest animal strap, and about midway between the first end and a second end of the body panel, a third connection point to the body panel and a fourth connection point to the body panel, the third connection point and the fourth connection point are mechanically coupled to an adjustable removable animal chest or abdomen strap, and at the second end of the body panel, and about opposite to the first end, a lower animal abdomen or belly strap.

The body panel can include at a first end of the body panel, a hanging loop.

The at least one strap can include a release buckle at a first end and a bar buckle at an opposite end.

A wearable fluid delivery system can include a body panel, the body panel including a plurality of connection points. A fluid dispenser can include a taut pocket defined by at least one taut surface and a portion of a surface of the body panel. The taut pocket is adapted to receive a fluid bag. A stretch pocket is disposed within the taut pocket. The stretch pocket is defined between a surface of at least one stretch panel and about a common portion of the surface of the body panel, the stretch pocket adapted to receive a pressure infusion bag disposed within. At least one shoulder strap extends from a first end of the body panel and releasably couplable to a first side of the body panel. At least one waist strap extends from a second side of the body panel and releasably couplable to the first side of the body panel.

A fluid dispenser and body panel assembly includes a body panel. A taut pocket is defined by at least one taut surface and a portion of a surface of the body panel, the taut pocket adapted to receive a fluid bag. A stretch pocket is disposed within the taut pocket, the stretch pocket defined between a surface of at least one stretch panel and about a common portion of the surface of the body panel. The stretch pocket is adapted to receive a pressure infusion bag disposed within.

The taut surface causes a taut wrap about both of the pressure infusion bag and the fluid bag.

The fluid bag can include an IV bag.

The stretch pocket can include a zipper opening along a first end of the body panel.

The taut pocket can include a zipper opening along a side of the body panel.

The fluid dispenser can include an inflation pump loop.

The taut pocket can include at least one fluid bag corner pocket.

The taut pocket can include at least one bag hook anchored to the body panel.

The foregoing and other aspects, features, and advantages of the application will become more apparent from the following description and from the claims.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 is a drawing showing a perspective view of a fluid delivery system according to the Application;

FIG. 2A is a drawing showing a pressure infusion bag disposed adjacent to a fluid bag;

FIG. 2B is a drawing showing the inflated pressure infusion bag applying pressure to the fluid bag of FIG. 2A;

FIG. 3 shows another exemplary wearable portable fluid delivery system laid out flat;

FIG. 4A is a drawing showing an angled top view of exemplary shoulder and arm straps suitable for use with the wearable fluid delivery system systems of FIG. 1 and FIG. 3;

FIG. 4B is a drawing showing another angled top view of the shoulder and arm straps of FIG. 4A;

FIG. 4C is a drawing showing a side view of the shoulder and arm straps of FIG. 4A;

FIG. 5 is a drawing showing an exemplary implementation of a wearable portable fluid delivery system according to FIG. 1 with a fluid bag installed;

FIG. 6 is a drawing showing details of the fluid bag installation of FIG. 5;

FIG. 7 is a drawing showing a pump, valve, tube, flow meter, and arm strap;

FIG. 8 is a drawing showing an openable taut panel of a taut pocket as a mesh;

FIG. 9 is a drawing showing a fluid bag set into a taut pocket;

FIG. 10 is a drawing showing the outer surface of the stretch panel of the stretch pocket;

FIG. 11 is a drawing showing a pressure infuser bag inserted through a zipper opening of the stretch pocket;

FIG. 12 is another drawing showing a pressure infuser bag inserted through a zipper opening of the stretch pocket;

FIG. 13 is a drawing showing another view of a fluid bag set over an open taut pocket;

FIG. 14 is a drawing showing an openable taut panel being moved to a closed position;

FIG. 15 is a drawing showing the openable taut panel of FIG. 14 in a closed position; and

FIG. 16 is a drawing showing the openable taut panel of FIG. 15 being closed by a zipper;

FIG. 17 is a drawing showing the taut panel and see-through mesh panel of the taut pocket (fluid bag);

FIG. 18 is a drawing showing stretch pocket within the taut pocket;

FIG. 19 is a drawing showing an exemplary sling worn wearable fluid delivery apparatus;

FIG. 20 is a drawing showing an exemplary universal fluid delivery system;

FIG. 21 is a drawing showing an exemplary sling under arm with above elbow strap worn wearable fluid delivery system;

FIG. 22 is a drawing showing an exemplary bicep strap with above elbow strap worn wearable fluid delivery system;

FIG. 23 is a drawing showing an exemplary waist and upper leg straps worn wearable fluid delivery system;

FIG. 24 is a drawing showing an exemplary upper leg and above the knee straps wearable fluid delivery system;

FIG. 25 is a drawing showing an exemplary around the neck and waist straps wearable fluid delivery system;

FIG. 26 is a drawing showing an exemplary animal shoulder waist straps wearable fluid delivery system;

FIG. 27 is a drawing showing a universal fluid delivery system hanging from a fluid deliver stand by hanging loop;

FIG. 28A is a drawing showing an exemplary flat side release buckle, a type of snap lock buckle;

FIG. 28B is a drawing showing an exemplary single bar buckle, similar to a sliding lock buckle;

FIG. 28C is a drawing showing an exemplary double bar D-ring Buckle;

FIG. 28D is a drawing showing an exemplary metal insert-buckle;

FIG. 28E is a drawing showing an exemplary spring buckle;

FIG. 28F is a drawing showing an exemplary prong buckle;

FIG. 29A is a drawing showing an exemplary hook and loop fastener;

FIG. 29B is a drawing showing an exemplary snap on fastener;

FIG. 29C is a drawing showing an exemplary magnetic fastener;

FIG. 29D is a drawing showing an exemplary zipper fastener.

FIG. 29E is a drawing showing an exemplary adhesive stick on fastening material;

FIG. 29F is a drawing showing an exemplary hook and hook fastener;

FIG. 30A is a drawing showing an exemplary swivel “lobster claw” clasp hook.

FIG. 30B is a drawing showing an exemplary spring snap link.

FIG. 30C is a drawing showing an exemplary standard hook clasp.

FIG. 30D is a drawing showing an exemplary round swivel eye bolt snap.

FIG. 30E is a drawing showing an exemplary push gate snap hook, swivel;

FIG. 31 is a drawing showing another exemplary universal fluid delivery system;

FIG. 32 is a drawing showing a rear or back view of the universal fluid delivery system of FIG. 31; and

FIG. 33 shows an exemplary layered fluid dispensing assembly.

### DETAILED DESCRIPTION

As described hereinabove, fluid delivery therapy, including intravenous (IV) therapy, is the infusion of a liquid-based medication, or other substance, into a patient. IV therapy is a fluid infusion directly into a vein of a patient. From 2005-2014 there was an approximate average of 380 disasters and there are 76,000 deaths from disasters annually. Medical rescue operatives use both gravity fed IV systems to hydrate and restore electrolytes in their patients, and also pressure infuser bags to pump high amounts of fluid into a patient to raise their blood pressure, get oxygen to the central organs and brain, and so that the patient can maintain a stabilized condition until reaching the hospital.

In disaster situations, there has yet to be a fluid delivery system that has been able to effectively provide victims with a fluid delivery therapy in any environment or situation. Also, there has yet to be a fluid delivery system to more efficiently and effectively provide timely fluid delivery therapy to disaster victims.

Therefore, there is a need for a faster and simpler way for first responders, particularly medical technicians and other medical professionals in the field, to more effectively and more efficiently provide fluid delivery treatment to victims the field, such as to treat injuries associated in the aftermath of disasters.

Another problem is that there has also yet to be a portable fluid delivery system that is designed to keep the fluid delivery system protected in dangerous environments such as combat zones, civil unrest, or for regions physically devastated by natural disasters, including the hazards, for example, of sharp fragments from collapsed buildings and infrastructure.

Additionally, hospitals, home infusion, and infusions centers currently do not have a portable fluid delivery system that allows patients to live their lives normally, without feeling that they are tethered to the fluid delivery system that is treating them. In the US one in five people have an autoimmune disease and a common form of treatment is fluid delivery by infusion.

Mobile fluid delivery systems exist, however, there is not a wearable portable fluid delivery system that can use pressure infusion to produce a high flow rate for the patient, while still having easy access to the fluid bag itself. Also, existing fluid delivery systems are relatively heavy, which limits their portability. In hospitals and infusion centers, there has yet to be a wearable fluid delivery system that will allow the medical staff to replace the fluids quickly.

Also, what is needed is a wearable mobile intravenous system that can effectively use most, if not all of the fluid delivery products in the market today.

The Application describes a wearable fluid delivery system which meets the needs and solves the problems described hereinabove.

The Application describes a new wearable fluid delivery system that can attach to the side of either shoulder, waist, legs, or other portions of the body, and combinations thereof, to give patients fluid therapy through gravity-fed drip and/or pressure fed fluid delivery system using a pressure infuser bag.

While many applications of the new wearable fluid delivery apparatus of the Application will likely be for the delivery of fluids, more generally, the apparatus can be used to deliver any suitable fluid to the body. Other fluid delivery applications include, for example, blood transfusions, vitamin infusion, egg white treatments as a blood thickener, chemotherapy, bone marrow treatments, etc.

The detailed description of the Application is divided into two parts. Part 1 describes exemplary structures of the fluid dispensing assembly. Part 2 describes strap and fastener structures to secure the fluid dispensing assembly to the body of a human or animal.

Fluid dispensing assembly—Combinations of a taut pocket, a stretch pocket, and panels of the pockets as described in detail in Part 1 of the Application, including any tubing and pump components are referred to collectively hereinbelow as the “fluid dispensing assembly”. The fluid dispensing assembly of part 1 is generally attached to a body panel. In addition to forming a “backer” for the fluid dispensing assembly, the body panel provides connection points for the various strap configurations described in part 2.

The fluid dispensing assembly can accept an embedded pressure infuser bag that will allow medical professionals to maintain any desired flow rate for the patient. The ability to maintain a desired flow rate enables medical rescue in a state of emergency to quickly distribute fluids to patients in any physical orientation (e.g. lying, sitting, or at an unusual position as found), as fast as possible without being restricted by their terrain such as uneven surfaces, hills, water, etc. Also, because of the favorable ergonomics and weight of the wearable portable fluid delivery system of the Application, emergency patients can more easily move, or be moved. The wearable portable fluid delivery system of the Application allows for placement of a fluid bag within, even where the patient has already been connected to an IV.

The body of the wearable portable fluid delivery system can be made from soft-goods materials and can be adjusted or tailored to fit each patient and user. One exemplary body of a wearable portable fluid delivery system according to the Application includes two straps. One strap goes across the chest and around the back, and the other goes around the arm to secure it to the user. One or both of the straps can extend or stretch to the size needed for the patient's arm. The wearable portable fluid delivery system can cover from the neck down to the elbow and can be worn on either shoulder.

The wearable portable fluid delivery system can include a fluid holder with two layers attached to a body panel to hold and support both the fluid bag and the pressure infusion bag, each in a separate layer. The first layer adjacent to the body can hold the pressure infusion bag. The first layer can be made with a stretchable material to compensate for the expanding pressure infusion bag. The second layer can hold the fluid bag and tubes. The second layer can made from a taut material with an optional see-through portion (e.g. a see through center portion), to ensure that the fluid bag will be squeezed against the expanding first layer (taut), and so that medical professionals can see the fluid levels of the bag (transparent or translucent panel, or any other suitable see through structure, such as, for example, a mesh or an opening).

The second layer of the wearable portable fluid delivery system can optionally have a zipper, hook and loop fastener (e.g. Velcro™), or any other suitable fastener or attachment means that can hold the fluid Bag within the fluid delivery system. The wearable portable fluid delivery system of the Application with an openable fastener allows medical professionals to easily replace a fluid bag for a patient that is already attached to into the wearable portable fluid delivery system, so that patient can now become fully mobile while receiving their treatment.

The wearable portable fluid delivery system of the Application can be made from any suitable non-metallic materials, metallic materials (e.g. fasteners and/or buckles), and combinations thereof, and needs no electronics to provide a patient with any desired flow rate through the use of the pressure infuser bag and a flow regulator.

The embedded pressure infuser bag along with a flow regulator and of the wearable portable fluid delivery system of the Application will speed up medical rescue in all emergency situations and dangerous environments (such as war, flood, earthquake, etc.) by being able to give the victims any desired flow rate from the fluid bag, while keeping the patient mobile or mobile ready. These dangerous environments can also compromise the fluid bag and the pressure infusion bag of the fluid delivery system. Therefore, in some embodiments, there can be a further third layer made of a hard material that will cover the other two layers, optionally with a see-through center, to ensure that there is no damage to either the fluid bag or the pressure infusion bag while still being able to see the water level of the fluid bag. The third layer can also be attached to the body panel of the wearable portable fluid delivery system and can be secured by any suitable means that is not easily opened, however which can be relatively easily opened by a medical professional when the fluid bag needs to be replaced. This way combat medics out in the field will be able to stabilize soldiers with fluids faster than ever before, through the embedded pressure bag system of the wearable portable fluid delivery system of the Application.

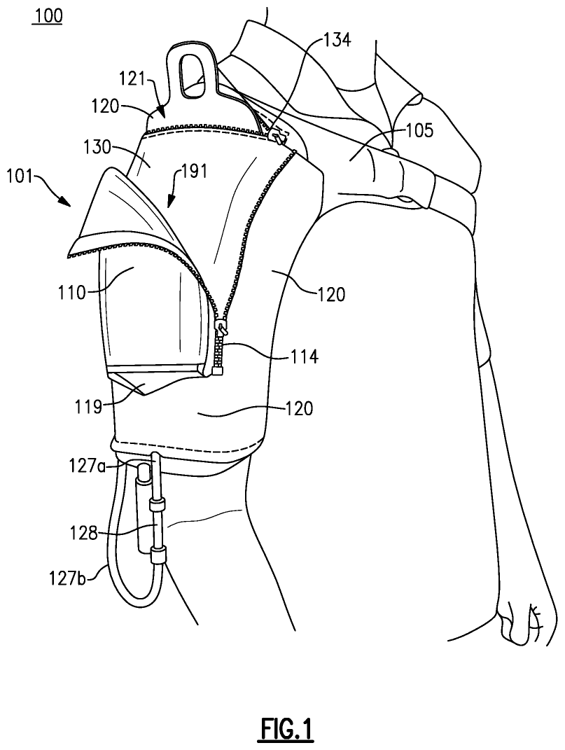

FIG. 1 shows an exemplary wearable portable fluid delivery system 100 according to the Application, worn on the body. Body panel 105 of the wearable portable fluid delivery system 100 can be made from a lightweight breathable material where the body panel 105 can wraps around one shoulder and arm, stopping at about the neck. The body panel 105 can be made of any suitable soft-goods material, or any other material that can hold the other components. In the example of FIG. 1, a strap wraps around the opposite side of the body. The strap is described in more detail hereinbelow.

Exemplary wearable portable fluid delivery system 100 includes two pockets (121, 191) as a stretch pocket 121 disposed within a taut pocket 191.

The stretch pocket 121 is adapted to receive an inflatable pressure infusion bag 120. Taut pocket 191 is adapted to receive any suitable type of fluid bag (fluid bag not shown in FIG. 1). Stretch pocket 121 (the pressure infusion bag pocket) is defined by stretch panel 130 of any suitable stretch type material. The exemplary stretchable wrap of stretch panel 130 (wraps part way around a pressure infusion bag 120) is sewn to the body panel 105 on both sides, and along the bottom, except where there is an opening for the inflation tube 127 (shown as sections 127a, 127b, about a pressure gauge 128 in FIG. 1). Stretch pocket 121 is also defined by an outer surface of body panel 105 which is behind the stretch panel 130 (i.e. where panel 130 has been sewed to the body panel 105). Stretch pocket 121 can be closable by a zipper 134.

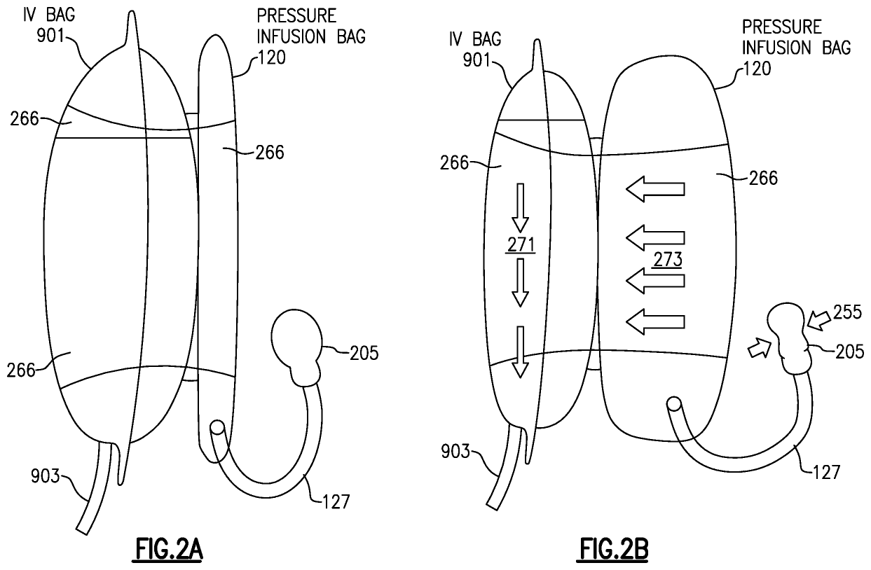

Taut pocket 191 includes the stretch pocket 121 disposed within. Taut pocket 191 is defined by taut panel 120 and openable taut panel 110. Exemplary taut panel 120 is also sewn along both side edges and the bottom edge (with openings for tubes) to the body panel 105. Any suitable fluid bag can be placed under the openable taut panel 110 between the inside surface of the openable taut panel 110 and the outside surface of stretch panel 130. Openable taut panel 110 closes and attaches to taut panel 110 by any suitable means, such as by zipper 114. Once zipper 114 is closed, taut panel 110, openable taut panel 110 and the outer surface of the body panel 105 between the stitched edges of taut panel 110 form a taut wrap 266 (FIG. 2A, FIG. 2B) defining about a constant volume within, because the taut surfaces expand relatively little. Note that while typically of a soft material, at least the body panel 105 portion which joins to one or more taut panels 110 is itself about taut because that part of the body panel 105 becomes part of an overall envelope of a taut wrap as described in more detail hereinbelow. The total volume within the envelope or taut wrap is relatively constant, while the volume defined within the stretch panel 130 varies with the inflation of the bladder of the pressure infusion bag 120. Note that the stretch pocket shares about a common outer surface portion of the body panel 105 where the sides of the stretch panel are sewn to the body panel 105, typically just inside of where the sides of the taut panel 120 are sewn to the body panel, so that both of the stretch pocket and the taut pocket share about a common portion of body panel 105. The stretch pocket could also be formed by attaching a more complete pre-assembled pocket or sock within the taut pocket and attached by any suitable means (e.g. adhesive, glue, sewing, etc. and combinations thereof) to the body panel 105. However, a pre-assembled pocket or sock should still be sewn or otherwise attached to the body panel 105 along at least two separate seams, so that the pressure bag does not significantly deform around the fluid bag and the taught wrap (e.g. an overall sock) does not deform the taught capture volume away from the body panel, and so that the pressure bag acts against the surface of the relatively stiff body panel 105 to most efficiently transfer pressure to a fluid bag in the taught pocket for best fluid flow rates from the fluid bag to a patient. As will be better understood in the detailed description that follows, merely attaching a taut enclosure to a body panel is insufficient, because then the pressure bag would not act uniformly against the relatively stiff body panel, rather the sock would deform. That is why both of the stretch pocket and the taught pocket about the stretch pocket are typically formed by two different panels which are joined by at least two different outer seams to the body panel which may or may not overlap, the stretch panel seams typically just inside of the outer taught panel seams (note that one side of the outer taught pocket side seam is typically one side of an openable closer such as a zipper or Velcro™ on a strip sewn to the body panel).

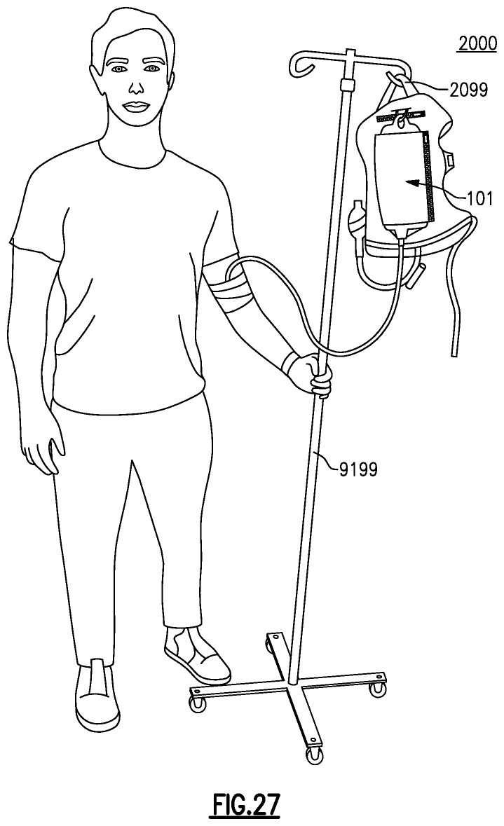

The fluid dispenser 101 of the wearable portable fluid delivery system according to the Application, includes stretch pocket 121, taut pocket 191, a portion of the body panel 105 adjacent to both pockets, related tubing, pumps, covers, optional labels, label holders, and/or triage pouches, etc. The fluid dispenser 101 can also include a loop at or near a first end of the body panel 105 (See for example, FIG. 27, 2099), so that the wearable portable fluid delivery system can be optionally hung on a fluid delivery or IV hook or pole.

FIG. 2A and FIG. 2B show a simplified operation of a wearable portable fluid delivery system according to the Application. The taut wrap 266 encloses both of the pressure infusion bag 201 and the fluid bag 901. In the example of FIG. 2A and FIG. 2B, a manual squeeze ball pump is used to inflate the pressure infusion bag 201. As shown in FIG. 2A, when a full fluid bag is first inserted into the taut pocket 191 of the exemplary wearable portable fluid delivery system, the full fluid bag has a largest full volume. The fluid bag lies adjacent to the pressure infusion bag 120, between an inner surface of openable taut panel 110 and an outer surface of stretch panel 130. The pressure infusion bag 120 lies within the stretch panel 130 between an inner surface of the stretch panel 130 and an outer surface of the about common portion of body panel 105 between the seams where the edges of the stretch panel 130 are sewn to the body panel 105.

FIG. 2B shows how the squeeze ball 205 can be manually compressed (arrows 255) to inflate the pressure infusion bag 201 via tube 127. Because the fluid bag 901 is held against the pressure inflation bag 201 by the taut wrap 266, the pressure infusion bag 201 imparts force 273 on the side of the fluid bag 901, causing a pressure flow 271 of the fluid from the fluid bag 901 via the fluid line tube 903 to the patient wearing the wearable portable fluid delivery system.

As noted hereinabove, FIG. 2A and FIG. 2B are simplified merely to illustration aspects of the principle of operation of a pressure infusion bag used in combination with a fluid delivery bag. However, the literal physical structure of FIG. 2A and FIG. 2B has several problems. On problem is that in reality, if the pressure bag 226 is simply placed next to the fluid delivery bag (e.g. IV bag 901), the pressure bag would wrap around the fluid delivery bag when inflated. Also, while the overall taut wrap is relatively inelastic, the shape of an overall taut wrap would change with the pressure of the pressure infusion bag limiting the efficiency of transfer from the pressure bag to the fluid bag.

It was realized that to provide the most efficient transfer of pressure from the pressure infusion bag to the fluid delivery bag, the pressure infusion bag should act against a common panel. A solution of the universal wearable fluid delivery system described by the Application is to load the pressure infusion bag into a stretch pocket which is formed by attaching the edges of the stretch pocket to a stiffer inelastic body panel. The body panel provides a relatively stiff surface area which is at least equal to, or more typically larger than the surface area of the side of the pressure infusion bag. The stretch pocket so formed against the body panel means that the pressure infusion bag applies pressure to the fluid delivery bag by acting against the relatively large and stiff surface of the body panel. Also, it was realized that by providing a separate stretch pocket within the taught pocket, the pressure infusion bag does not deform by expanding around the fluid delivery bag. Such deformation around the fluid bag can lead to a less efficient transfer of pressure from the pressure infusion bag to the fluid delivery bag.

Another problem illustrated by the simplified principle of operation drawings of FIG. 2A and FIG. 2B is that with only one common outer taut wrap, it can be more difficult to change out the fluid delivery bag. It was realized that by use of two different structural pockets, the separate stretch pocket within an openable outer taut pocket, that it can be much easier to replace the fluid delivery bag. Such replacements might be caused by a practitioner calling for a different type of fluid, or more likely for quick and efficient removal of an empty fluid delivery bag for replacement by a fresh filled fluid delivery bag, without need to adjust the pressure of the pressure infusion bag.

Thus, as described in more detail hereinbelow, the fluid dispenser of a universal wearable fluid delivery system according to the Application includes a taut pocket defined by at least one taut surface and a portion of a surface of the body panel. The taut pocket is adapted to receive a fluid bag. A stretch pocket is disposed within the taut pocket. The stretch pocket is defined between a surface of at least one stretch panel and about a common portion of the surface of the body panel. The stretch pocket is adapted to receive a pressure infusion bag disposed within. That way, the pressure infusion bag acts against the body panel which has a surface area about the same as, or more typically larger than the surface area of the side of the pressure bag, as defined by the seams of the taut bag to the body panel where the pressure bag is contained within the stretch pocket for an improved and more efficient transfer of pressure from the pressure infusion bag to the fluid delivery bag, thus improving the rate of flow of fluid from the fluid bag to the patient. Moreover, the taut panel opens, such as by a zipper or Velcro™ closure at least along one side of the taut panel, making for an easy and fast replacement of the fluid bag, regardless of the pressure of the pressure infusion bag at the time of fluid bag replacement.

The pressure infusion bag is disposed within stretch pocket 121. Stretch panel 130 can be made of stretchy fabric or any other suitable material that is able to stretch with the expansion and contraction of the pressure infusion bag held within. The back of stretch pocket 121 closest to the body panel 105 can be attached to the body panel 105 through traditional sewing methods or any other suitable attachment means. This back portion of stretch pocket 121 is relatively not stretchable (relatively taut) because this portion of the body panel 105 is shared about in common with the back of taut pocket 191 to form the overall taut wrap 266 about both of the pressure infusion bag (stretch pocket 121) and the fluid bag (taut pocket 191, which includes disposed within taut pocket 191, and shares the about common “taut” back wall along a portion of body panel 105). There can also be additional components inside of stretch pocket 121 (not shown in FIG. 1) that hold the pressure infusion bag in place to ensure that the pressure infusion bag does not shift. There can also be corner pockets to help secure a fluid bag within the outer taut pocket 191.

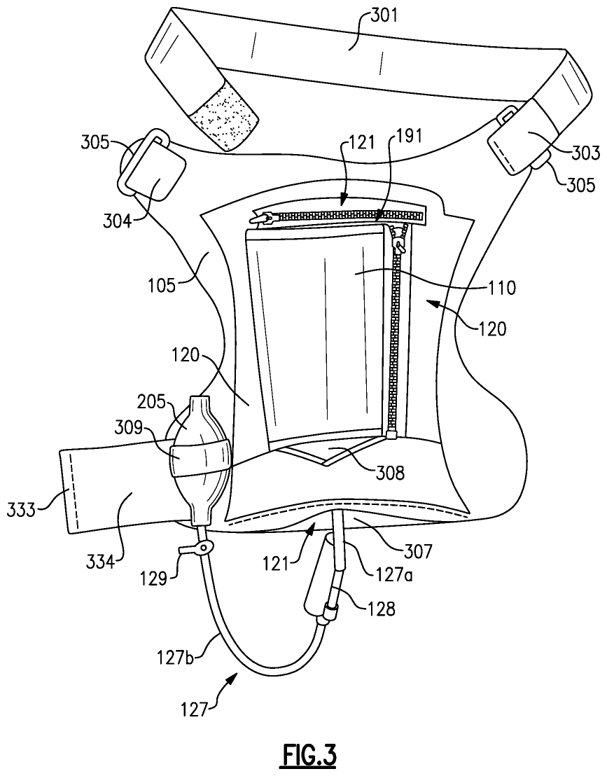

FIG. 3 shows the exemplary wearable portable fluid delivery system of FIG. 1 laid out flat. Stretch pocket 121 (pump infusion bag) has an opening 307 on the bottom end of pocket 121 that allows for the tubing 127 of the pressure infusion bag to pump 205 to be exposed. A pressure gauge 128 can be optionally installed in tubing 127, such as, is shown disposed between tube 127a and tube 127b in FIG. 3. Opening 307 can be made openable or closeable by use of any suitable method for latching including Velcro™, zipper, etc. Pump tube 127 exits stretch pocket 121 through the opening 307. Pump 205 typically hangs or comes out of the pressure infusion bag as connected by tube 127. The pump holder 309 is typically a stretchy (stretchable) fabric strap that holds the pump taught against the arm of the patient. However, holder 309 can be made of any other suitable structure or method for holding a pump up against the body such as, for example, a clip, hook, etc., and any combination thereof. Holder 309 can be located anywhere on the wearable portable fluid delivery system that allows a user to easily and efficiently access the pump.

Openable taut panel 110 can be made from any suitable see-through fabric 306 for viewing fluid levels while the wearable portable fluid delivery system is in use. The exemplary see-through material 306 of FIG. 1 and FIG. 3 is a mesh type material, however see-through material 306 can also be made from any other suitable see-through material such as, for example, plastic, glass, etc. Below the see-through material 306, there is an opening 308 that allows the tubing to be connected to the fluid bag. In the exemplary wearable fluid delivery system of FIG. 3, there are corner pockets on either side of opening 308 to capture the lower right and left corners of a fluid bag. Opening 308 can also be a hole made in a surface of any suitable material such as, for example, plastic, rubber, etc.

In the exemplary wearable fluid delivery system of FIG. 3, there is an elastic Velcro™ arm strap 334 that allows the wearable portable fluid delivery system to comfortably fit around a user's arm and to hold the body panel 105 in place. While the exemplary method for attachment is Velcro™, any other suitable structure or method for holding the body panel 105 around the user's arm can be used, such as, for example, hooks, slots, zippers, etc., and any combination thereof.

Openable taut panel 110 of taut pocket 191 (for the fluid bag) is shown as closed by a zipper 114 in the exemplary wearable portable fluid delivery system 100 of FIG. 1. However, openable taut panel 110 can be closed about a fluid bag by zipper, or any other suitable means of attachment that ensures that the pressure infusion bag is replaceable and held within layer one 10 such as Velcro™ adhesive, etc. Similarly, any suitable closing means can be used for the closure of stretch pocket 121 (for the pressure infusion bag) in place of zipper 134.

Taut pocket 191 also includes an opening 308 for one or more tubes from the fluid bag. Any suitable opening can be used ranging from an opening defined by stitched seems about a bottom edge of openable taut panel 110, to a bottom surface of any suitable material with an opening or hole in the bottom surface (not shown in FIG. 1).

Taut pocket 191 can be adapted to hold any suitable fluid delivery bag (typically 100 ml or below) as well as any other fluid delivery components such as related tubing. Taut pocket 191 allows the fluid bag to be placed in an upright position so as to deliver fluids to a patient wearing a wearable portable fluid 100. The panels of taut pocket 191 (e.g. taut panel 120, openable taut panel 110, and the common portion of body panel 105) can be made of any suitable taut material such as, for example, fabric, plastic, metal, etc. Such materials only need be taut enough so that when the pressure infusion bag expands, the fluid bag will discharge a fluid under the pressure imposed by the about adjacent pressure infusion bag.

There can also be additional components disposed within taut pocket 191, such as, for example the two lower corner pockets on either side of opening 308, to ensure that the fluid bag 901 does not shift and to relieve pressure on or around the arm. In the exemplary wearable portable fluid delivery system 100, the fluid bag 901 is held within taut pocket 191 via zipper 114. However, any other suitable method of attachment that ensures that the fluid bag is replaceable and held securely within taut pocket 191, such as, for example, Velcro™, adhesive, etc.

The components of the exemplary wearable portable fluid delivery system of FIG. 1, FIG. 3, are typically sewn together, however such components can be attached using any suitable means, such as, for example, adhesive, buckle, screw, hooks, other suitable fasteners, and any combination thereof.

There can also be a third layer of one or more panels (not shown in FIG. 1). By layer, we mean to define a successive outer wrap in the sense of a layer of clothing, however, as with the stretch pocket and the taut pocket, an outer protective layer would also typically share a common outer surface of the body panel 105. In other words, the layers are somewhat analogous to rings of tree trunk, however they are not concentric, but rather offset as sharing, or adjacent to, about a common portion of the body panel 105. The third layer can assist the taut panels to maintain the about constant volume within the taut pocket, or a third layer can be added only to protect the components within, where the overall taut wrap is still provided by the outer panels of the taut pocket.

A third outer protective layer can made of any suitable relatively hard material such as, for example wood, metal, plastic etc. that can cover the other two layers with an optional see-through center viewing port, to ensure that there is no damage to either the fluid delivery system or the pressure infusion bag while still being able to see the fluid level of the fluid bag. The third layer can also be attached to the body panel, and can be secured with a method that is not easily opened but can be opened by a medical professional to replace the bag. The third layer can similarly be attached to the body panel 105 by any suitable seams, sewn seams, or fasteners including those described hereinabove, such as, for example, by hook, Velcro™, hinge, etc., and any combination thereof.

Generally, the pressure infusion bag is closer to the shoulder with the about adjacent fluid bag on the outside, farthest from shoulder. As such, the see-through panel allows for easy viewing of the fluid level in the fluid bag. However, those skilled in the art will appreciate that otherwise the order of the bags is unimportant, where there are similar ways to reverse the order of the bags and still show, for example, a see-through side view of the fluid bag, without departing from the overall teachings of the wearable portable fluid delivery system taut wrap concept (e.g. FIG. 2A, FIG. 2B) of the Application.

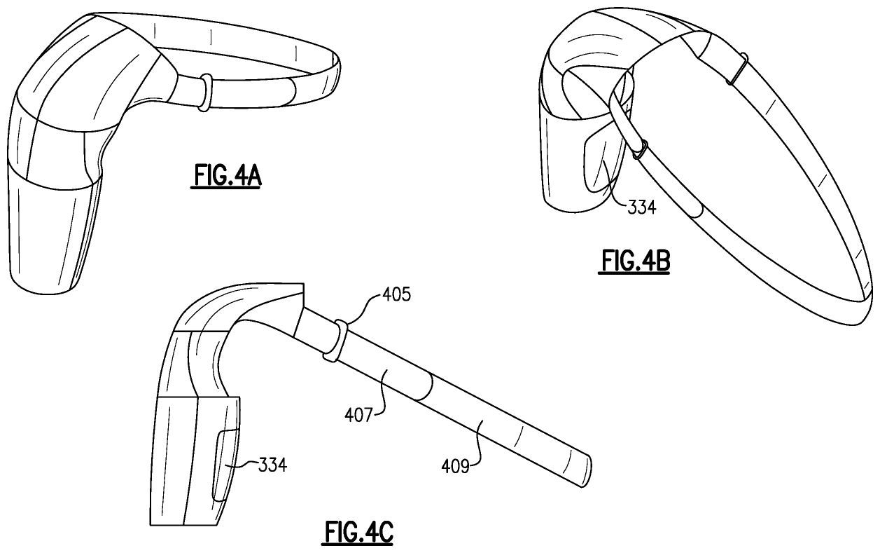

FIG. 4A to FIG. 4C show an exemplary shoulder strap which supports the fluid dispenser 101 on an outer shoulder of the patient. The exemplary shoulder strap also supports the patient's shoulder itself for comfort. The shoulder strap can be relatively quickly and easily put on a patient by use of the two straps. Strap 409 goes under the opposite armpit and across the chest. In the example, strap 409 wraps around a buckle 405 of strap part 407 and secures by use of Velcro™ However, any other suitable joining or buckling techniques, such as, for example, hooks or adhesives or any combination thereof can be used. A second strap 334, (See also FIG. 3, 334) wraps under the bicep and also uses Velcro™ to secure the second strap 334 around the patient's arm. The second strap can be attached using similar or any other suitable fastening structure or method. The bicep strap 334 can also stretch or otherwise be adaptable to compensate for different sized individual's arms. The exemplary straps are made with elastics, soft goods, and Velcro™ however can alternatively be made from any suitable materials that can be worn by the body comfortably.

Any suitable fluid drip chambers can be used as standard use in the art. Any suitable fluid flow control cylinders, devices, modules, etc. can be used in the fluid bag line 903.

Exemplary Implementation

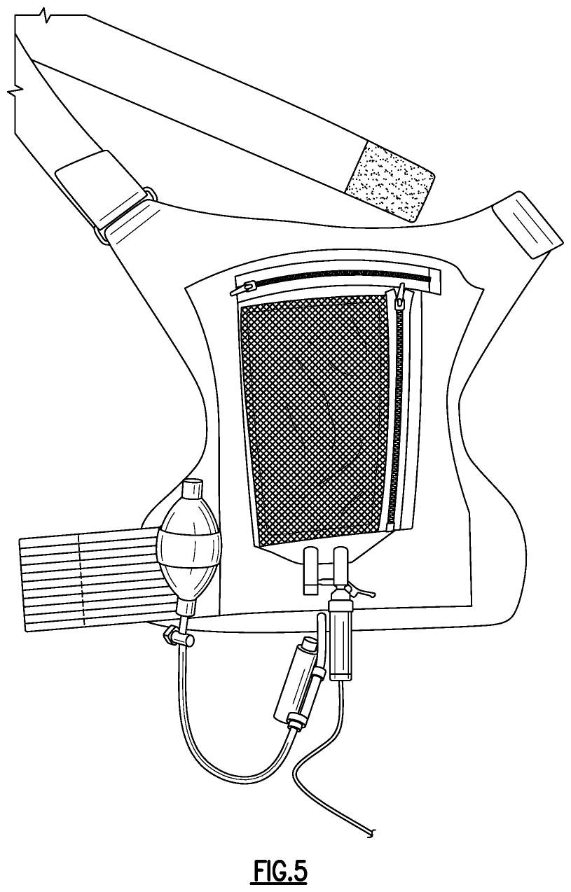

FIG. 5 to FIG. 16 are drawings showing more detail of an exemplary implementation fabricated according to the wearable portable fluid delivery system 100 of FIG. 1 and FIG. 3.

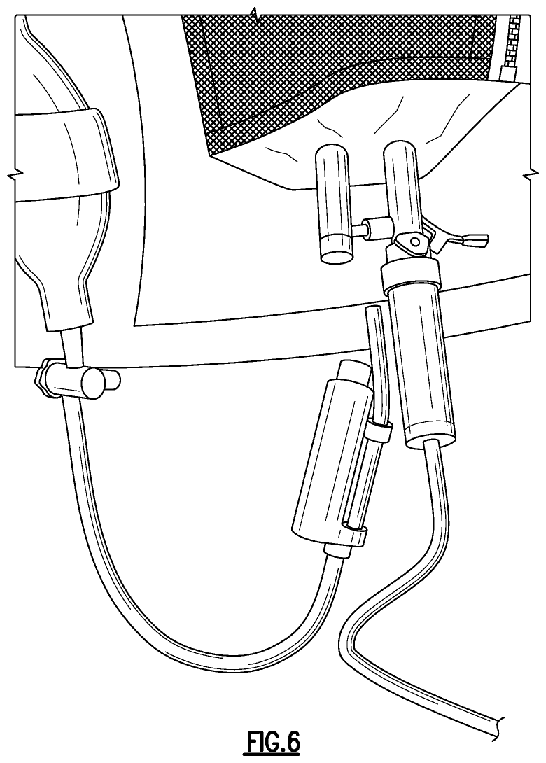

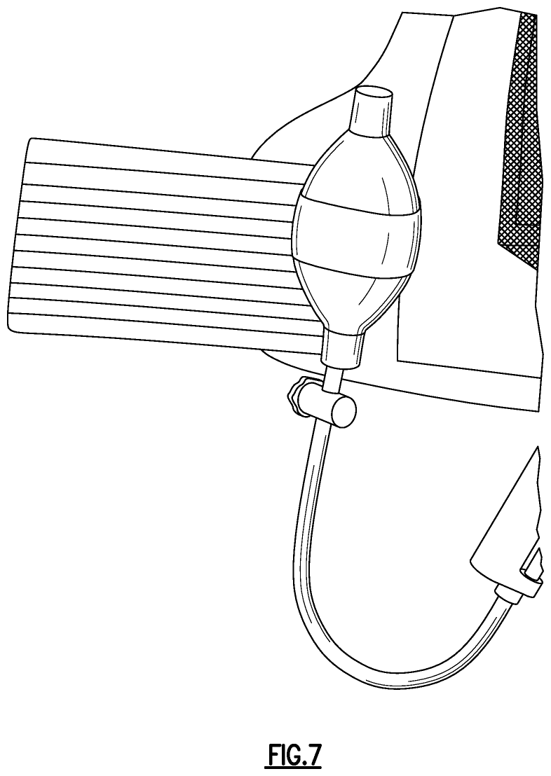

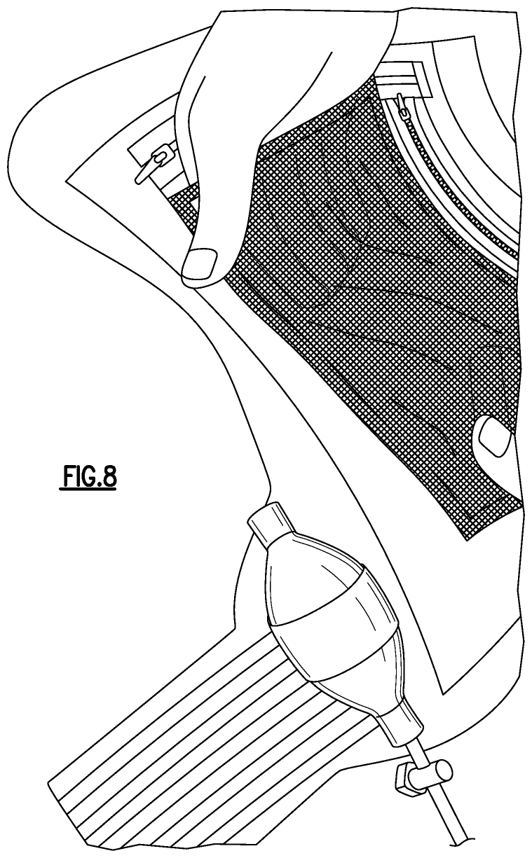

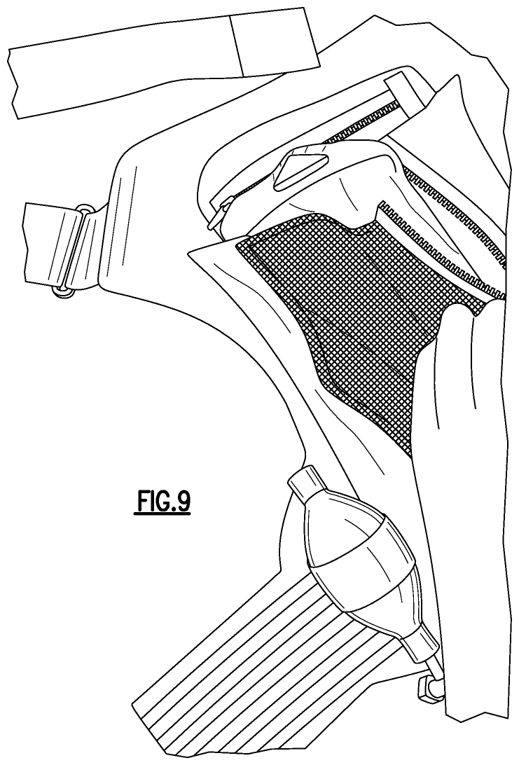

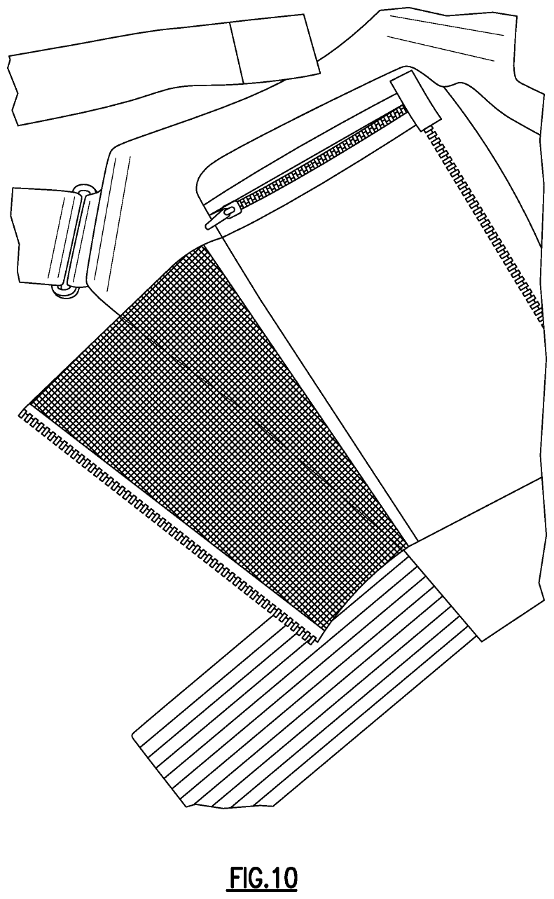

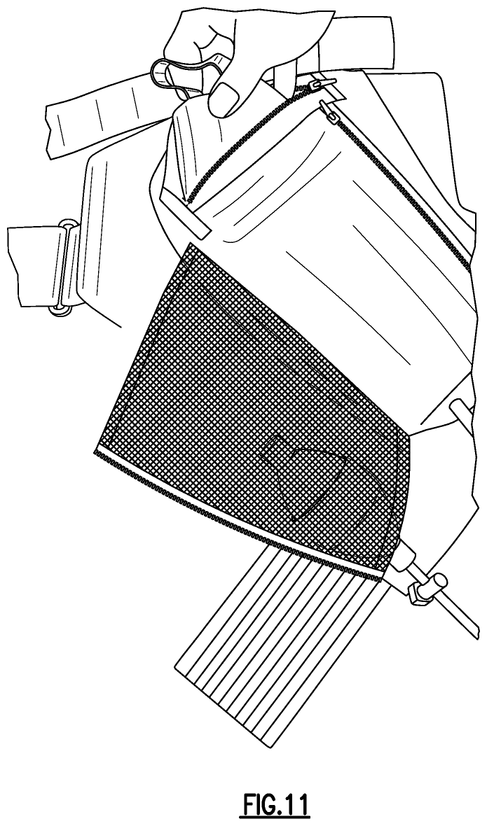

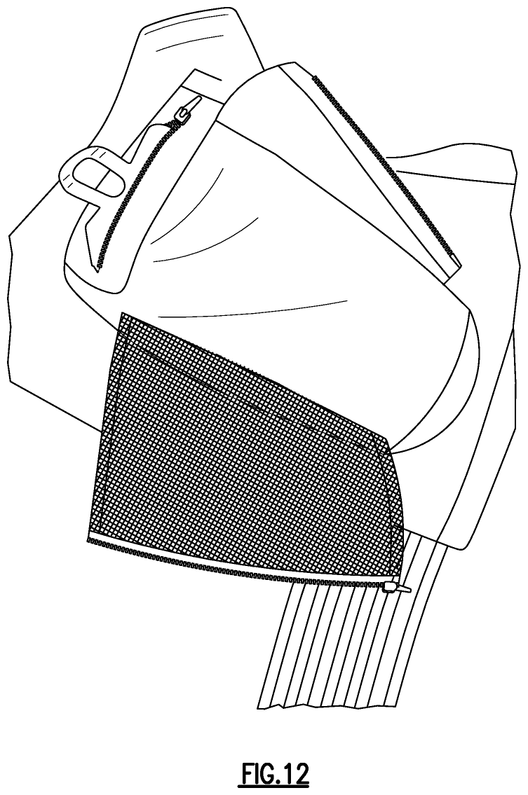

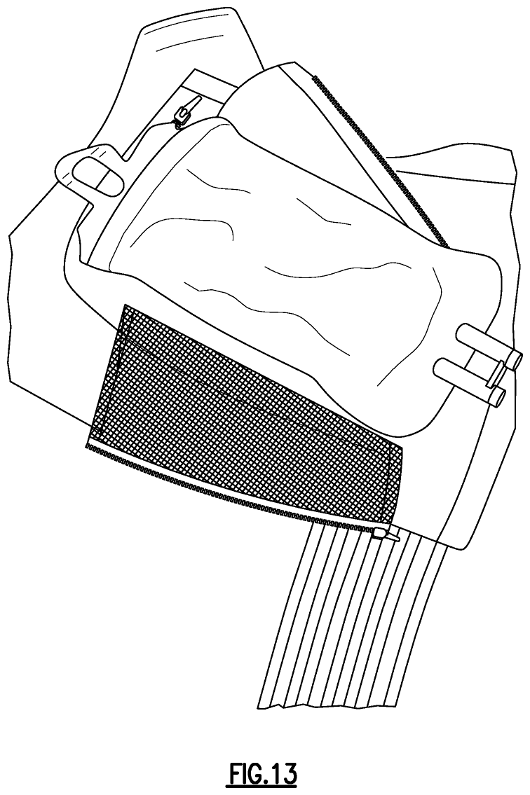

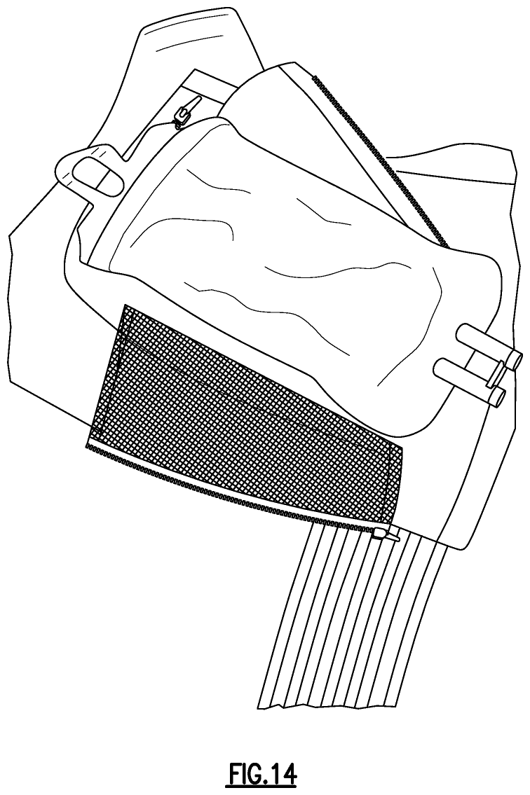

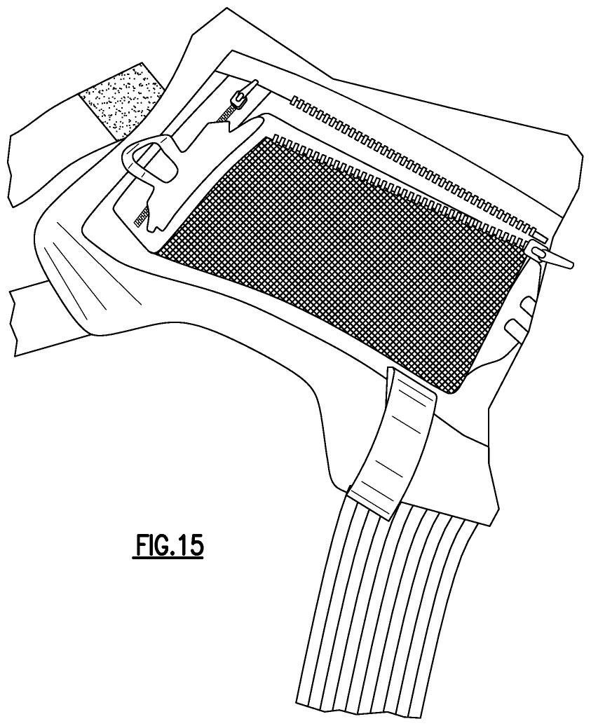

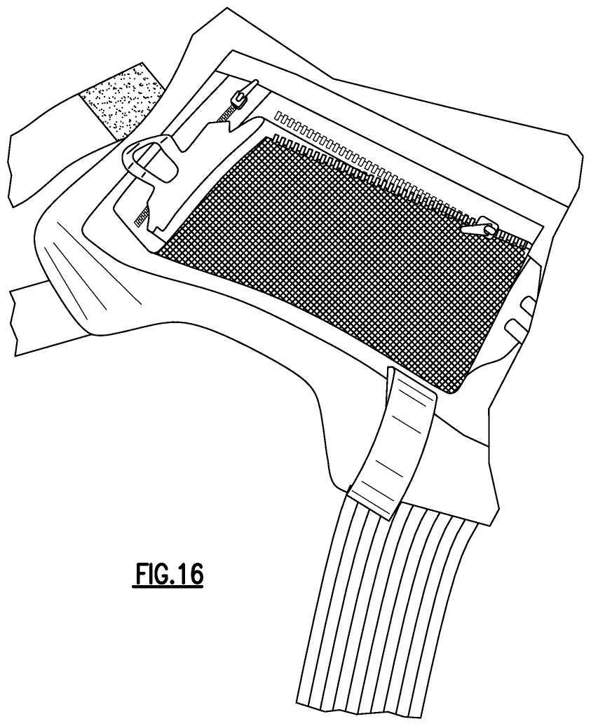

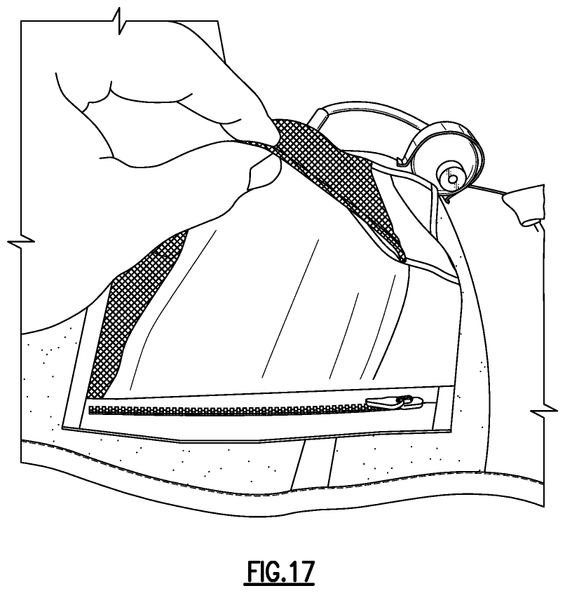

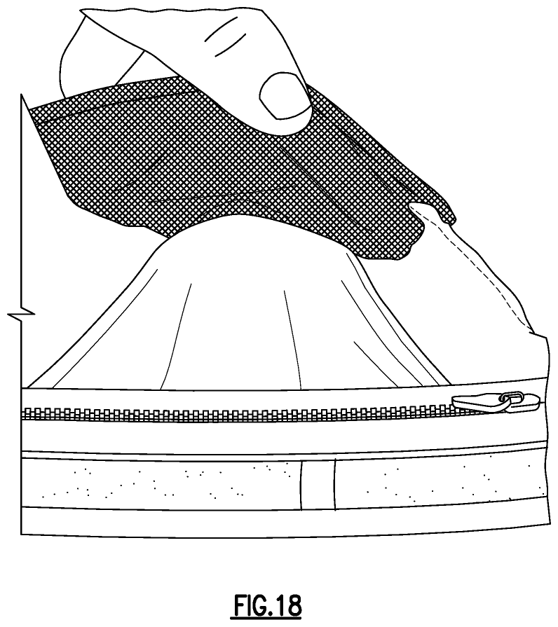

FIG. 5 is a drawing showing an exemplary implementation of a wearable portable fluid delivery system according to FIG. 1 with a fluid bag installed. FIG. 6 is a drawing showing details of the fluid bag installation of FIG. 5. FIG. 7 is a drawing showing a pump, valve, tube, flow meter, and arm strap. FIG. 8 is a drawing showing an openable taut panel of a taut pocket as a mesh. FIG. 9 is a drawing showing a fluid bag set into a taut pocket. FIG. 10 is a drawing showing the outer surface of the stretch panel of the stretch pocket. FIG. 11 is a drawing showing a pressure infuser bag inserted through a zipper opening of the stretch pocket. FIG. 12 is another drawing showing a pressure infuser bag inserted through a zipper opening of the stretch pocket. FIG. 13 is a drawing showing another view of a fluid bag set over an open taut pocket. FIG. 14 is a drawing showing an openable taut panel being moved to a closed position. FIG. 15 is a drawing showing the openable taut panel of FIG. 14 in a closed position. FIG. 16 is a drawing showing the openable taut panel of FIG. 15 being closed by a zipper. FIG. 17 is a drawing showing the taut panel and see-through mesh panel of the taut pocket (fluid bag). FIG. 18 is a drawing showing stretch pocket within the taut pocket.

Stretch pocket—The stretch pocket shown in the examples are generally made as inset pockets (a pocket within a pocket), where the stretch pocket is created by the space between an inside surface of a stretch panel and a portion of the surface of the body panel. However, the stretch pocket disposed within the taut pocket can be of any suitable type of pocket construction. For example, a complete pocket of a stretch material can be inserted into the taut pocket against the body panel. In such cases where the stretch pocket is a complete pocket disposed within the taut pocket and placed against the body panel, the side of the stretch pocket adjacent to the body panel can be attached to the body panel by any suitable fastening means, including, for example, sewing, stitching, gluing, adhesive, hook and fasteners (e.g. Velcro™) etc.

In summary, and generally with reference to the exemplary structures of FIG. 1 and FIG. 3, a fluid dispenser 101 and body panel assembly includes body panel 105. A taut pocket 191 is defined by at least one taut surface (e.g. taut panel 110) and a portion of a surface of the body panel 105, the taut pocket 191 adapted to receive a fluid bag. A stretch pocket 121 is disposed within the taut pocket 191, the stretch pocket 121 defined between a surface of at least one stretch panel 130 and about a common portion of the surface of the body panel 105.

Part 2—Securing the fluid dispensing assembly to a human or animal body

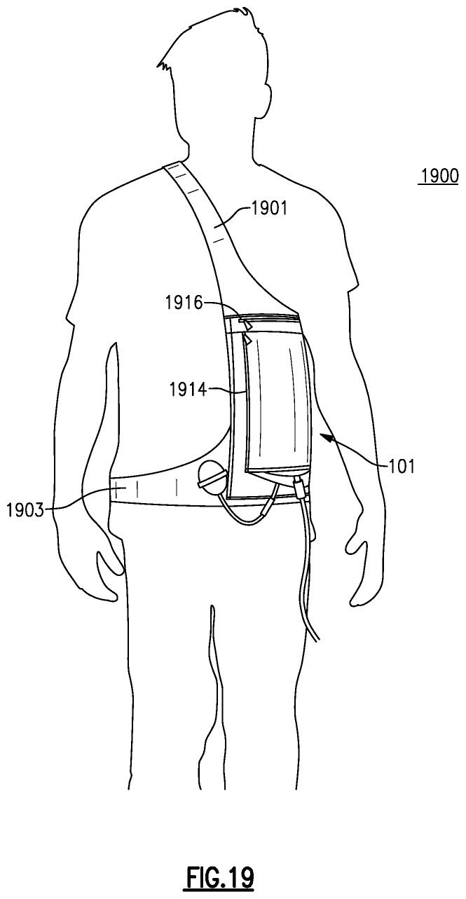

Sling over shoulder with waist strap—FIG. 19 is a drawing showing an exemplary sling worn wearable fluid delivery apparatus which includes a fluid dispensing assembly, fluid dispenser 101, as described in detail in Part 1 of the Application. The exemplary sling worn wearable fluid delivery system 1900 includes wearable fluid dispenser 101, shoulder strap 1901 and waist strap 1903. In this exemplary structure of FIG. 19, both of the shoulder strap 1901 and waist strap 1903 are either extensions of the material of the body panel 105, or either or both of the shoulder strap 1901 and waist strap 1903 can be sewn or stitched on one end to the body panel 105. The other side of each of the shoulder strap 1901 and waist strap 1903 can be removably attached to corresponding attachment points on the body panel 105 (corresponding attachment points not visible in FIG. 19). Suitable detachable or removable attachment devices are described in more detail hereinbelow. In the exemplary wearable fluid dispenser 101 of FIG. 19, zipper 1914 provides access to the taut pocket for inserting or removing a fluid bag, and zipper 1916 provides access to the stretch pocket for inserting or removing a pressure infusion bag. Note that the pressure infusion bag is typically reusable, at least for a duration of use by one patient, while the fluid bag can be changed out as often as needed for additional or different type of fluid infusion.

Universal Fluid Delivery System

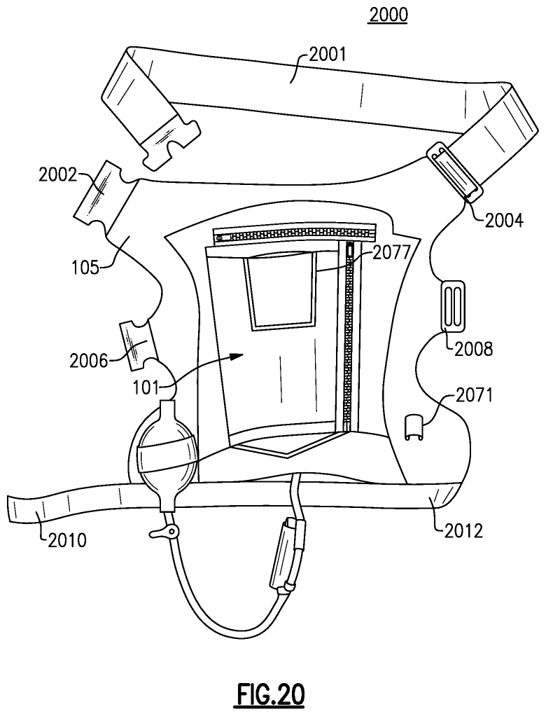

FIG. 20 is a drawing showing an exemplary universal fluid delivery system 2000 based on a wearable fluid dispenser 101 as described hereinabove. The universal nature of the fluid delivery system 2000 is provided by a number of strap connection points, upper connection points, 2002, 2004, middle connection points, 2006, 2008, and a bottom strap 2010 and strap closure 2012. In the example of FIG. 20, upper connection point 2002 is a snap lock buckle receptacle, which is configured to receive a snap lock buckle plug of a strap. Upper connection point 2008 is a slip lock buckle fastener configured to accept a strap end. Middle connection points 2006 is a snap lock buckle receptacle, which is configured to receive a snap lock buckle plug of a strap. Middle connection point 2008 is a slip lock fastener configured to accept a strap end. bottom strap 2010 has a portion which can join strap closure 2012 by an overlap, such as by hooks and loops (e.g. Velcro) or adhesive on one or both parts of the strap 2010 overlap with the strap closure 2012.

The universal nature of the fluid delivery system 2000 of FIG. 20 is based on the presence of three or more connection points, and not on the particular style or type of each connection. The drawings which follow show several ways that a fluid dispenser 101 can be attached to the body of a human or animal according to a wearable fluid delivery system 2000. Different types of exemplary suitable buckles are described in more detail hereinbelow. It is unimportant what type of plug, receptacle, hook, adhesive, etc., is used to affix each side of each strap to the universal wearable fluid dispenser 101. Also, any suitable strap, including strings, rope, or wire can be alternatively used for any of the straps. The exemplary straps can be provided in different sizes for different wearing styles, or in one relatively long universal length which can be tied off or can be cut to any desired length during or following attachment to an animal or person.

Generally, and with reference to the example of FIG. 20, a universal wearable fluid delivery system 2000 includes a body panel 105. The body panel 105 includes a plurality of connection points (e.g. 2002, 2004, 2006, 2008, 2012). A fluid dispenser 101 includes a taut pocket defined by at least one taut surface and a portion of a surface of the body panel. The taut pocket is adapted to receive a fluid bag. A stretch pocket is disposed within the taut pocket. The stretch pocket is defined between a surface of at least one stretch panel and about a common portion of the surface of the body panel. The stretch pocket is adapted to receive a pressure infusion bag disposed within. At least one strap (e.g. 2001) is coupled to the body panel via two connection points of the plurality of connection points (e.g. 2002, 2004). The at least one strap 2001 is to secure the fluid dispenser to a body of a human or animal patient.

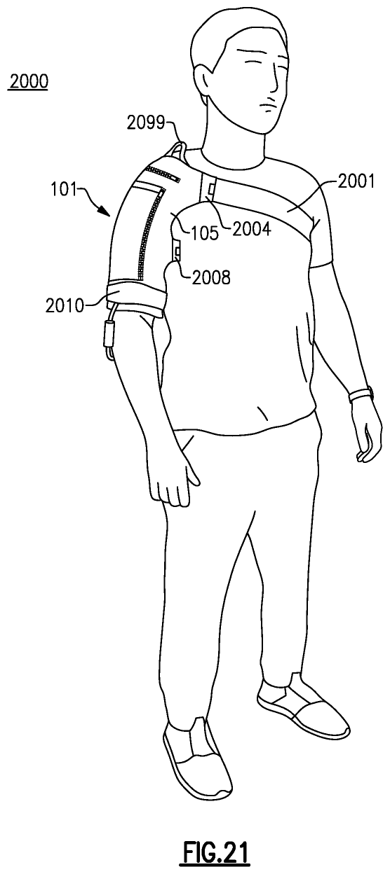

Sling under arm with above elbow strap—FIG. 21 is a drawing showing an exemplary sling under arm with above elbow strap worn wearable fluid delivery system 2000 which includes a fluid dispensing assembly as described in Part 1 of the Application. The exemplary Sling under arm with above elbow strap universal fluid delivery system 2000 includes wearable fluid dispenser 101, under the shoulder strap 2001 and above the elbow strap 2010. The under the shoulder strap 2001 can be coupled using any suitable type of connector between upper connection points, 2002, 2004 (FIG. 20, FIG. 21). The above the elbow strap 2010 can join strap closure 2012 by an overlap, such as by a hook and loop (e.g. Velcro) or adhesive on one or both parts of the strap 2010 overlap with the strap closure 2012. Connectors and/or buckles could be used in place of the overlapping strap joinder.

Optional system hanging loop 2099 allows for hanging the universal fluid delivery system 2000 from any suitable hook when not wearing the system, such as for example, a hook of an fluid bag stand, such as an IV stand or fluid bag or IV hook extending from a patient gurney, stretcher, bed, or wall, etc. Hanging loop 2099 can also be used for quick access storage, such as from a hook of a storage cabinet, storage closet, or any other suitable storage or dispensing facility.

Generally, and with reference to the example of FIG. 21, a universal wearable fluid delivery system 2000 can include at a first end of the body panel, a first connection point 2004 to the body panel 105 and a second connection point to the body panel (e.g. FIG. 20, 2002, not visible, FIG. 21), the first connection point 2004 and the second connection point mechanically coupled to an adjustable removable across a chest and under arm strap 2001, and at a second end of the body panel, opposite to the first end, an above an elbow strap (2010).

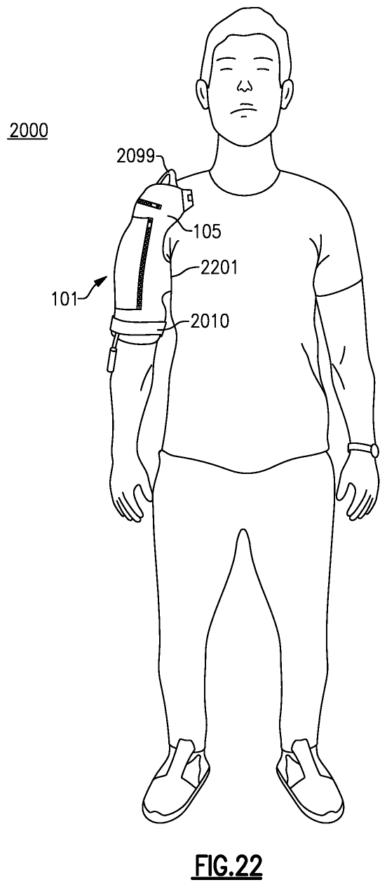

Bicep strap with above elbow strap—FIG. 22 is a drawing showing an exemplary bicep strap with above elbow strap worn wearable fluid delivery system which includes a fluid dispensing assembly as described in Part 1 of the Application. The exemplary bicep strap with above elbow strap universal fluid delivery system 2000 includes wearable fluid dispenser 101, bicep strap 2201 and above the elbow strap 2012. The above the elbow strap 2012 can join strap closure 2012 by an overlap, such as by a hook and loop (e.g. Velcro) or adhesive on one or both parts of the strap 2010 overlap with the strap closure 2012. Connectors and/or buckles could be used in place of the overlapping 2010/strap closure 2012 strap joinder.

Generally, and with reference to the example of FIG. 22, a universal wearable fluid delivery system 2000 can include about midway between a first end and a second end of the body panel, a third connection point (not visible FIG. 22, e.g. FIG. 20, 2006) to the body panel and a fourth connection point (not visible FIG. 22, e.g. FIG. 20, 2008) to the body panel, the third connection point and the fourth connection point mechanically coupled to an adjustable bicep strap 2201, and at a second end of the body panel, opposite to the first end, an above the elbow strap.

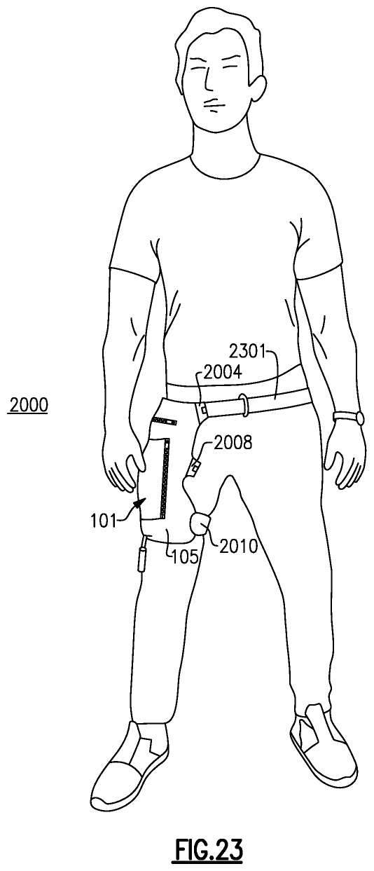

Waste and upper leg straps—FIG. 23 is a drawing showing an exemplary waist and upper leg strap worn wearable fluid delivery system which includes a fluid dispensing assembly as described in Part 1 of the Application. The exemplary waist and upper leg straps universal fluid delivery system 2000 includes fluid dispenser 101, waist strap 2301 and upper leg strap 2012. The waist strap 2301 can be coupled using any suitable type of connector between upper connection points, 2002, 2004 (FIG. 20, FIG. 23). The above the upper leg strap 2010 can join strap closure 2012 by an overlap, such as by a hook and loop (e.g. Velcro) or adhesive on one or both parts of the strap 2010 overlap with the strap closure 2012. Connectors and/or buckles could be used in place of the overlapping 2010/strap closure 2012 strap joinder.

Generally, and with reference to the example of FIG. 23, a universal wearable fluid delivery system 2000 can include at a first end of the body panel, a first connection point 2004 to the body panel 105 and a second connection point to the body panel 2002 (e.g. FIG. 20, 2002, not visible, FIG. 23), the first connection point 2004 and the second connection point mechanically coupled to an adjustable removable waist strap 2301, and at a second end, opposite to the first end, an upper leg strap 2010.

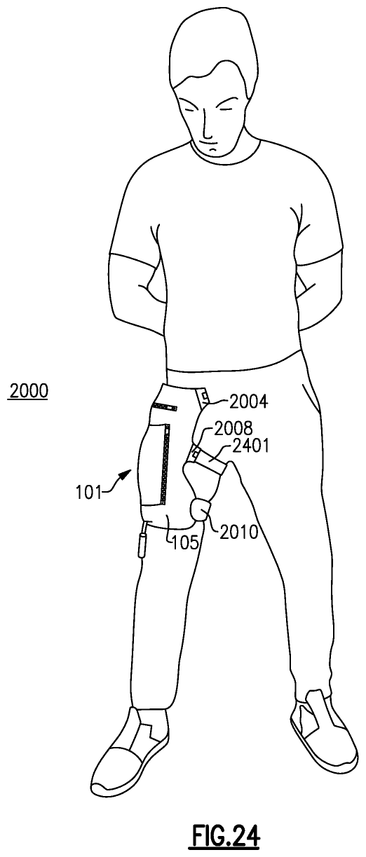

Upper leg and above the knee straps—FIG. 24 is a drawing showing an exemplary upper leg and above the knee straps which includes a fluid dispensing assembly as described in Part 1 of the Application. The exemplary Sling upper leg and above the knee straps universal fluid delivery system 2000 includes fluid dispenser 101, upper leg strap 2401 and above the knee strap 2012. The upper leg strap 2401 can be coupled using any suitable type of connector between upper connection points, 2006, 2008 (FIG. 20, FIG. 24). The above the knee strap 2012 can join strap closure 2012 by an overlap, such as by a hook and loop (e.g. Velcro) or adhesive on one or both parts of the strap 2010 overlap with the strap closure 2012. Connectors and/or buckles could be used in place of the overlapping strap joinder.

Generally, and with reference to the example of FIG. 24, a universal wearable fluid delivery system 2000 can include about midway between a first end and a second end of the body panel, a third connection point to the body panel and a fourth connection point to the body panel, the third connection point 2008 and the second connection point 2006 (e.g. FIG. 20, 2006, not visible, FIG. 24), mechanically coupled to an adjustable removable upper leg or crotch strap 2401, and at the second end of the body panel, opposite to the first end, an above the knee strap 2010.

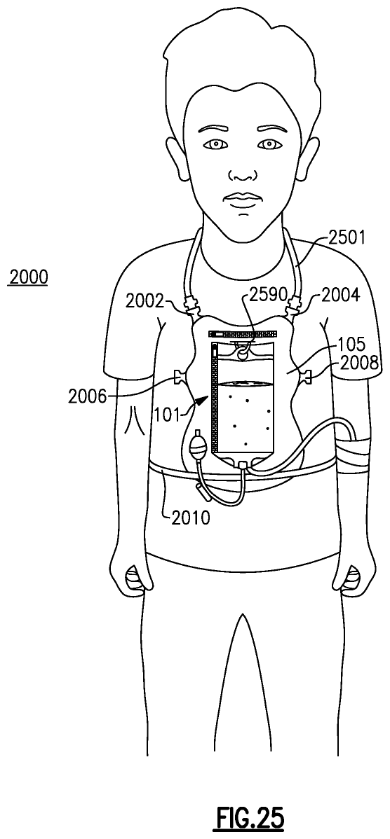

Around the neck and waist straps—FIG. 25 is a drawing showing an exemplary around the neck and waist straps which includes a fluid dispensing assembly as described in Part 1 of the Application. The exemplary around the neck and waist straps universal fluid delivery system 2000 includes fluid dispenser 101, neck strap 2501 and waist strap 2010. The neck strap 2501 can be coupled using any suitable type of connector between upper connection points, 2002, 2004 (FIG. 20, FIG. 25). The waist strap 2010 can join strap closure 2012 by an overlap, such as by a hook and loop (e.g. Velcro) or adhesive on one or both parts of the strap 2010 overlap with the strap closure 2012. Connectors and/or buckles could be used in place of the overlapping strap joinder.

Generally, and with reference to the example of FIG. 25, a universal wearable fluid delivery system 2000 can include at a first end of the body panel 105, a first connection point 2002 to the body panel and a second connection point 2004 to the body panel, the first connection point and the second connection point mechanically coupled to an adjustable removable around a neck strap 2501, and at a second end of the body panel, about opposite to the first end, a lower torso or waist strap 2010.

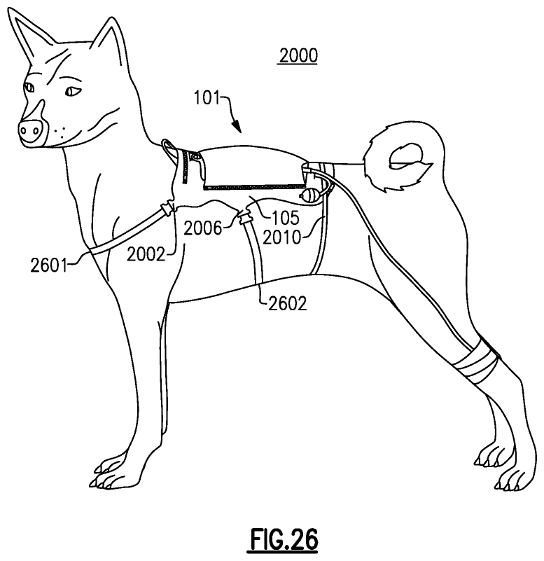

Animal shoulder waist straps—FIG. 26 is a drawing showing an exemplary animal shoulder waist straps which includes a fluid dispensing assembly as described in Part 1 of the Application. The exemplary animal (such as, for example, a dog as shown in FIG. 26) shoulder and waist straps universal fluid delivery system 2000 includes wearable fluid dispenser 101, shoulder strap 2601, waist strap 2602, and waist strap 2010. The waist straps 2601, 2602 can be coupled using any suitable type of connector between upper and middle connection points, 2002, 2004, 2006, and 2008 (FIG. 20, FIG. 26). The waist strap 2010 can join strap closure 2012 by an overlap, such as by a hook and loop (e.g. Velcro) or adhesive on one or both parts of the strap 2010 overlap with the strap closure 2012. Connectors and/or buckles could be used in place of the overlapping strap joinder.

Generally, and with reference to the example of FIG. 26, a universal wearable fluid delivery system 2000 can include at a first end of the body panel 105, a first connection point to the body panel and a second connection point to the body panel, the first connection point 2002 and the second connection point (e.g. FIG. 20, 2004, not visible, FIG. 26), mechanically coupled to an adjustable removable over a shoulder forechest animal strap 2601, and about midway between the first end and a second end of the body panel, a third connection point 2006 to the body panel and a fourth connection point (e.g. FIG. 20, 2006, not visible, FIG. 26), to the body panel, the third connection point and the fourth connection point mechanically coupled to an adjustable removable animal chest or abdomen strap 2602, and at the second end of the body panel, about opposite to the first end, a lower animal abdomen or belly strap 2010.

As described hereinabove, the pressure bag acts against the relatively stiff body panel of about a same surface area or larger than the side of the pressure bag. However, while relatively stiff, especially compared to the elastic material of the stretch panel, the body panel can still be flexible enough to conform in whole or in part to the surfaces of the body to which it is pressed against by the various strap configurations and patient wear configuration described by the Application. Note that even where the body panel is flexible enough to conform in whole or in part to the body of the patient, where the straps affix the body panel against the surface of the body of the patient, the body of the patient can also help to enhance the stiffness of the body panel of the worn universal wearable fluid delivery system.

Stand hook—FIG. 27 is a drawing showing a universal fluid delivery system 2000 hanging from a fluid deliver stand 9199 by hanging loop 2099.

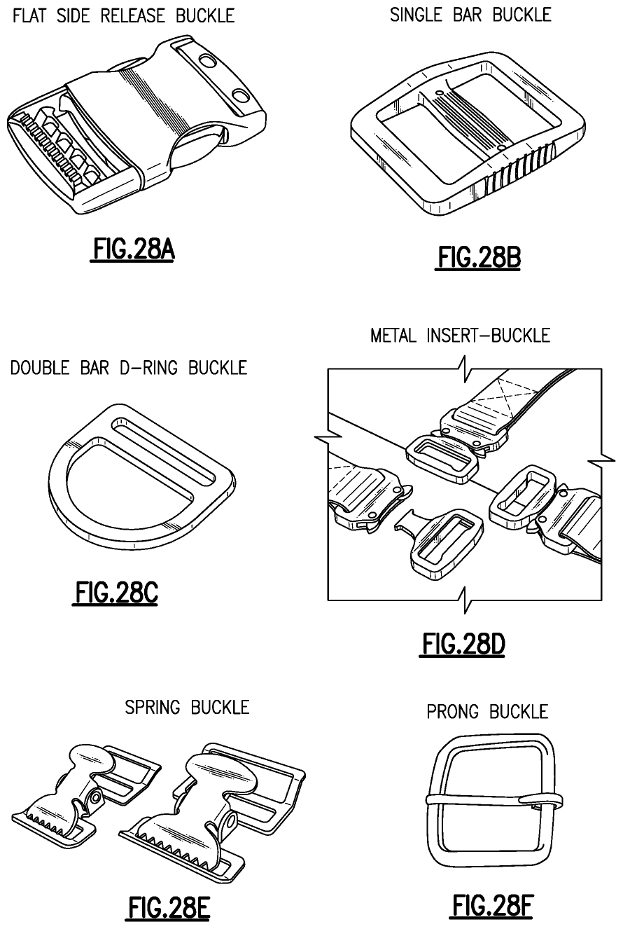

Buckles—Any suitable connectors such as any suitable buckles can be used for any of the straps used with a fluid delivery system according to the Application. FIG. 28A to FIG. 28F are drawings showing exemplary suitable buckles. FIG. 28A is a drawing showing an exemplary flat side release buckle, a type of snap lock buckle. FIG. 28B is a drawing showing an exemplary single bar buckle, similar to a sliding lock buckle. FIG. 28C is a drawing showing an exemplary double bar D-ring Buckle. FIG. 28D is a drawing showing an exemplary metal insert-buckle. FIG. 28E is a drawing showing an exemplary spring buckle. FIG. 28F is a drawing showing an exemplary prong buckle. Buckles can be made from any suitable material, typically a plastic, thermoplastic, nylon, metal, and combinations thereof.

While some end point connections are shown as buckles (e.g. FIG. 20, 2002, 2004, 2006, 2008, and others (e.g. FIG. 20, strap 2010 with adhesive, Velcro, etc. connection to 2012), any of the end point connections can be extensions of the body panel 105 (e.g. continuous straps from the same or similar material), sewn on or otherwise permanently fastened at one end, any suitable buckles, fasteners, and combinations thereof.

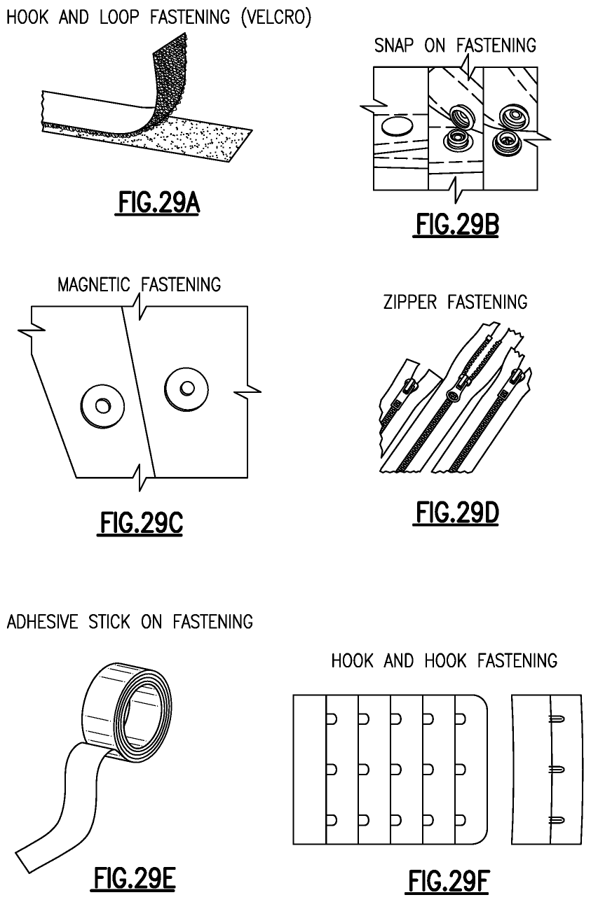

Fasteners—Any suitable fasteners can alternatively be used for any of the straps used with a fluid delivery system according to the Application. Also, fasteners can be used to close either or both of the taut pocket, stretch pocket, and any other openable panels. FIG. 29A to FIG. 29F are drawings showing exemplary suitable fasteners. FIG. 29A is a drawing showing an exemplary hook and loop fastener (e.g. Velcro). FIG. 29B is a drawing showing an exemplary snap on fastener. FIG. 29C is a drawing showing an exemplary magnetic fastener. FIG. 29D is a drawing showing an exemplary zipper fastener. FIG. 29E is a drawing showing an exemplary adhesive stick on fastening material. FIG. 29F is a drawing showing an exemplary hook and hook fastener. Buckles can be made from any suitable materials, typically a plastic, thermoplastic, nylon, woven fabric, metal, and combinations thereof.

Straps—straps can be made of any suitable strap material, typically including woven fabrics, such as a woven nylon. Wire, plastic covered wire, strings, and metallic or non-metallic, lines, cords, ropes, etc. can be used in place of straps.

Body panel—The body panel can be made of any suitable material, including fabric, woven fabric, rubber, plastic, nylon, etc.

Ruggedized fluid delivery system options—In many expected applications, ranging from disaster areas and forest fire fighting to war zones and combat, ruggedized materials and techniques can be used. Ruggedized materials can be used, such as materials that can withstand military temperature ranges for both storage and use. Fabric panels can include ruggedized woven nylon, NOMEX type fabrics, KEVLAR, or any combination thereof. There can also be an additional hook or clasp to secure the fluid bag within the taut pocket.

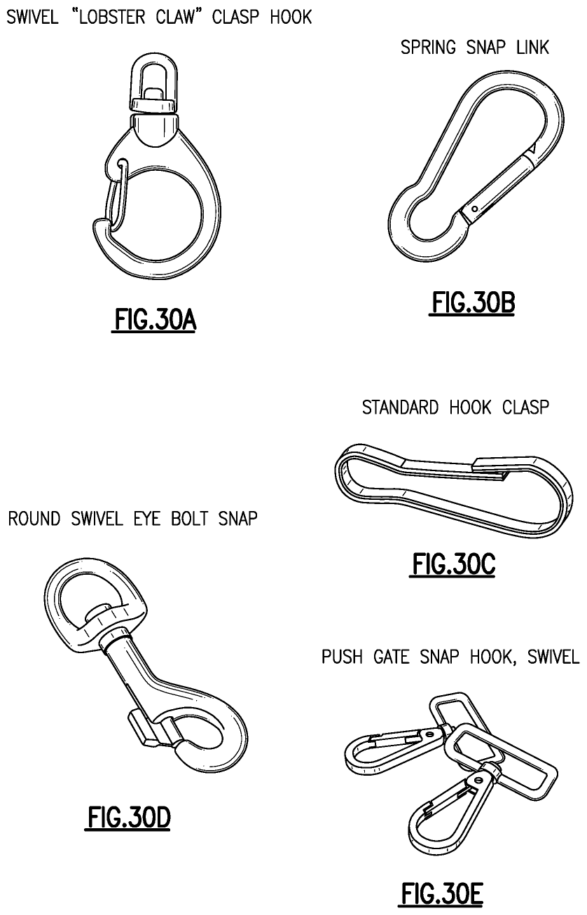

Hooks and clasps—Any suitable hooks can alternatively be used with a fluid delivery system according to the Application. FIG. 30A to FIG. 30F are drawings showing exemplary suitable hooks and clasps. FIG. 30A is a drawing showing an exemplary swivel “lobster claw” clasp hook. FIG. 30B is a drawing showing an exemplary spring snap link. FIG. 30C is a drawing showing an exemplary standard hook clasp. FIG. 30D is a drawing showing an exemplary round swivel eye bolt snap. FIG. 30E is a drawing showing an exemplary push gate snap hook, swivel. Fasteners can be made from any suitable materials, typically a plastic, thermoplastic, nylon, metal, and combinations thereof.

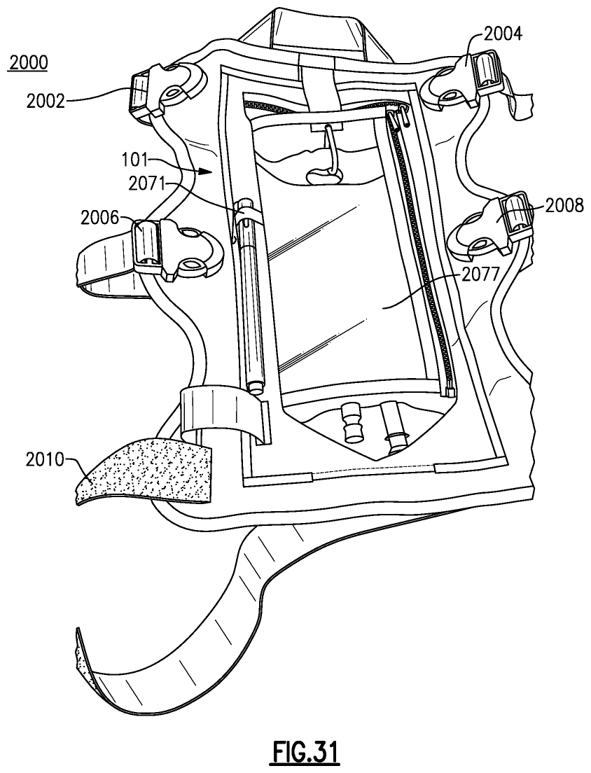

FIG. 31 is a drawing showing another exemplary universal fluid delivery system 2000. FIG. 31 shows an alternative configuration of upper connection point buckles, 2002, 2004, and middle connection point buckles, 2006, 2008. Also shown in FIG. 31, is a clear window pocket 2077, such as can be used for triage or treatment information, and a marker loop 2071 for a marker, pen, pencil, etc.

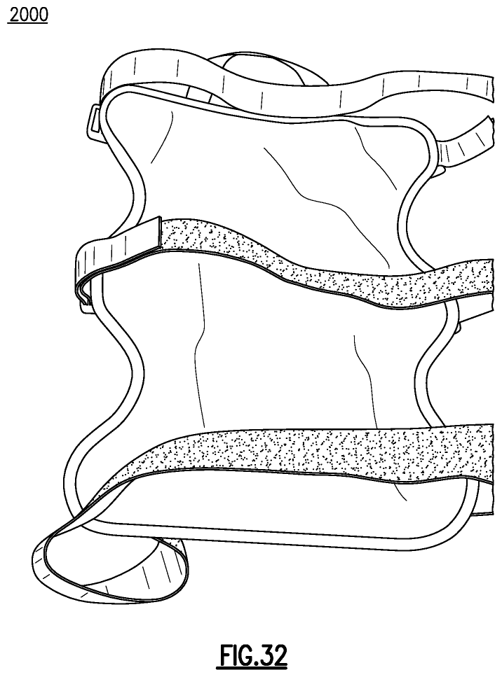

FIG. 32 is a drawing showing a rear or back view of the universal fluid delivery system 2000 of FIG. 31.

Fluid dispenser 101 can also include layers, of any suitable materials of varying compressibility, such as, for example, layers of neoprene.

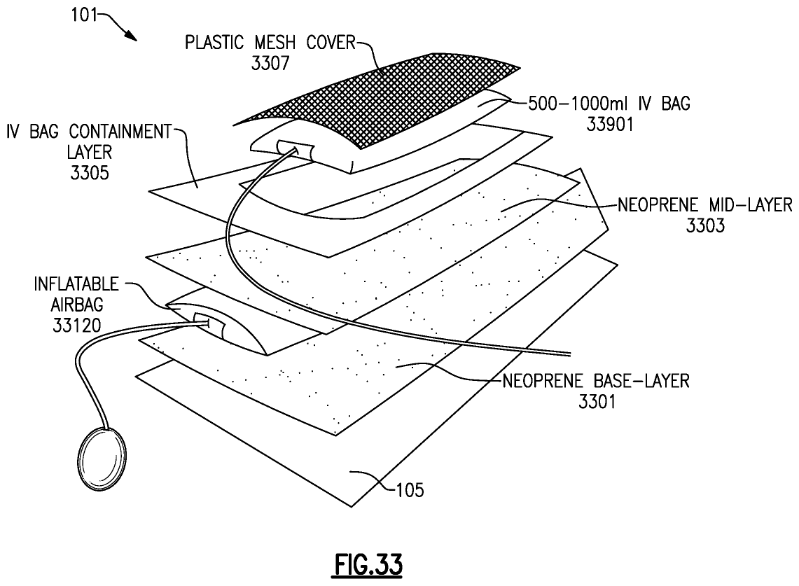

FIG. 33 shows an exemplary layered fluid dispenser 101. For simplicity, the outer fabric and fasteners which complete the stretch pocket and the taut pocket are understood to be present, however have been omitted for simplicity. Above the body panel 105 is a first neoprene-based layer 3301 at the back of the stretch pocket. A neoprene mid-layer 3303 is adjacent to the outer side of the stretch pocket. A fluid bag containment layer 3305 has a cutout to secure a fluid bag, such as, for example, an IV bag. A plastic mesh cover 3307 can cover the fluid bag similar to other exemplary fluid delivery systems described hereinabove.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A universal wearable fluid delivery system comprising:
a body panel, said body panel comprising a plurality of connection points;
a fluid dispenser comprising:
a taut pocket defined by at least one taut surface and a portion of a surface of said body panel, said taut pocket adapted to receive a fluid bag;
a stretch pocket disposed within said taut pocket, said stretch pocket defined between a surface of at least one stretch panel and about a common portion of said surface of said body panel, said stretch pocket adapted to receive a pressure infusion bag disposed within; and
at least one strap coupled to said body panel via two connection points of said plurality of connection points, said at least one strap to secure said fluid dispenser to a body of a human or animal patient.

2. The universal wearable fluid delivery system of claim 1, wherein said body panel comprises:
at a first end of said body panel, a first connection point to said body panel and a second connection point to said body panel, said first connection point and said second connection point mechanically coupled to an adjustable removable across a chest and under arm strap; and
at a second end of said body panel, opposite to said first end, an above an elbow strap.

3. The universal wearable fluid delivery system of claim 1, wherein said body panel comprises:
about midway between a first end and a second end of said body panel, a third connection point to said body panel and a fourth connection point to said body panel, said third connection point and said fourth connection point mechanically coupled to an adjustable bicep strap; and
at a second end of said body panel, opposite to said first end, an above an elbow strap.

4. The universal wearable fluid delivery system of claim 1, wherein said body panel comprises:
at a first end of said body panel, a first connection point to said body panel and a second connection point to said body panel, said first connection point and said second connection point mechanically coupled to an adjustable removable waist strap; and
at said second end of said body panel, opposite to said first end, an above a knee strap.

5. The universal wearable fluid delivery system of claim 1, wherein said body panel comprises:
about midway between a first end and a second end of said body panel, a third connection point to said body panel and a fourth connection point to said body panel, said third connection point and said fourth connection point mechanically coupled to an adjustable removable upper leg or crotch strap; and
at said second end of said body panel, opposite to said first end, an above a knee strap.

6. The universal wearable fluid delivery system of claim 1, wherein said body panel comprises:
at a first end of said body panel, a first connection point to said body panel and a second connection point to said body panel, said first connection point and said second connection point mechanically coupled to an adjustable removable around a neck strap; and
at a second end of said body panel, about opposite to said first end, a lower torso or waist strap.

7. The universal wearable fluid delivery system of claim 1, wherein said body panel comprises:
at a first end of said body panel, a first connection point to said body panel and a second connection point to said body panel, said first connection point and said second connection point mechanically coupled to an adjustable removable over a shoulder forechest animal strap; and
about midway between said first end and a second end of said body panel, a third connection point to said body panel and a fourth connection point to said body panel, said third connection point and said fourth connection point mechanically coupled to an adjustable removable animal chest or abdomen strap; and
at said second end of said body panel, about opposite to said first end, a lower animal abdomen or belly strap.

8. The universal wearable fluid delivery system of claim 1, wherein said body panel comprises at a first end of said body panel, a hanging loop.

9. The universal wearable fluid delivery system of claim 1, wherein said at least one strap comprises a release buckle at a first end and a bar buckle at an opposite end.

10. A wearable fluid delivery system comprising:
a body panel, said body panel comprising a plurality of connection points;
a fluid dispenser comprising:
a taut pocket defined by at least one taut surface and a portion of a surface of said body panel, said taut pocket adapted to receive a fluid bag;
a stretch pocket disposed within said taut pocket, said stretch pocket defined between a surface of at least one stretch panel and about a common portion of said surface of said body panel, said stretch pocket adapted to receive a pressure infusion bag disposed within; and
at least one shoulder strap extending from a first end of said body panel and releasably couplable to a first side of said body panel; and
at least one waist strap extending from a second side of said body panel and releasably couplable to said first side of said body panel.

11. A fluid dispenser and body panel assembly comprising:
a body panel;
a taut pocket defined by at least one taut surface and a portion of a surface of said body panel, said taut pocket adapted to receive a fluid bag; and
a stretch pocket disposed within said taut pocket, said stretch pocket defined between a surface of at least one stretch panel and about a common portion of said surface of said body panel, said stretch pocket adapted to receive a pressure infusion bag disposed within.

12. The fluid dispenser of claim 11, wherein said taut surface causes a taut wrap about both of said pressure infusion bag and the fluid bag.

13. The fluid dispenser of claim 11, wherein said fluid bag comprises an IV bag.

14. The fluid dispenser of claim 11, wherein said stretch pocket comprises a zipper opening along a first end of said body panel.

15. The fluid dispenser of claim 11, wherein said taut pocket comprises a zipper opening along a side of said body panel.

16. The fluid dispenser of claim 11, further comprising an inflation pump loop.

17. The fluid dispenser of claim 11, wherein said taut pocket further comprises at least one fluid bag corner pocket.

18. The fluid dispenser of claim 11, wherein said taut pocket further comprises at least one bag hook anchored to said body panel.
